(19) United States
(12) Patent Application Publication
(10) Pub. No.: US 2004/0071741 A1
(43) Pub. Date: Apr. 15, 2004

(54) Use of silicone waxes containing ester functional groups for thickening oily media

(75) Inventor: Paul-Joel Derian, Lawrenceville, NJ (US)

Correspondence Address: NORMAN H. STEPNO, ESQUIRE, BURNS, DOANE, SWECKER & MATHIS, L.L.P., P.O. Box 1404, Alexandria, VA, 22313-1404, US

(73) Assignee: RHONE-POULENC CHIMIE, Cedex (FR)

(21) Appl. No.: 10/428,788

(22) Filed: May 5, 2003

Related U.S. Application Data
(63) Continuation of application No. 09/504,819, filed on Feb. 16, 2000, now abandoned.
Application No. 09/504,819 is a continuation of application No. 08/632,814, filed on Apr. 17, 1996, now abandoned.

(30) Foreign Application Priority Data
Apr. 18, 1995 (FR) 95 04582

(51) Int. Cl. A61K 9/00

(52) U.S. Cl. 424/400

(57) ABSTRACT

Use, for thickening oily media, of silicone waxes which have aliphatic fatty alcohol or acid ester functional groups of formula —(CH₂)ₙ—A-R, in which formula n is equal to at least 2, A an —OCO— or —COO—group and R a C₂₁-C₃₀ aliphatic group, more particularly those which have behenate functional groups —(CH₂)₃—O—CO—(CH₂)₂₀—CH₃, bonded directly to silicon atoms of the polyorganosiloxane chain.

Process for thickening oily media by addition of the said waxes to the said media.

Compositions, especially cosmetic ones (sun gels, deodorants), including the said media thus thickened.

The present invention relates to the use of silicone waxes containing aliphatic fatty alcohol or acid ester functional groups for thickening oily media or to a process for thickening oily media by addition of the said waxes to the said media, and to the oily media including a silicone wax containing aliphatic fatty alcohol or acid ester functional groups as thickening agent.

It has already been proposed to thicken oily media such as hydrocarbon oils, mineral oils, vegetable oils or cosmetic or pharmaceutical preparations with the aid of a polyorganosiloxane which has C₁₈-C₃₆ saturated aliphatic functional groups and optionally —(CH₂)ₓ—COOR functional groups, where R denotes a C₁-C₄ alkyl group, with x capable of ranging from 2 to 12, which functional groups are directly bonded to the silicon atoms of the polyorganosiloxane chain (U.S. Pat. No. 4,844,826).

Similarly, polyorganosiloxanes containing —R—COOR′ and optionally —R—OH functional groups, where R denotes a C₂-C₁₈ divalent aliphatic radical and R¹ a C₈-C₂₀ aliphatic radical, which radicals are directly bonded to the silicon atoms of the polyorganosiloxane chain, have been used for the preparation of cosmetic compositions for shaving, which compositions can be presented in the form of creams, of gels, of self-foaming gels, of aerosol foams or of bars (EP-A-0 376 820).

The Applicant Company has found that silicone waxes which have functional groups of formula —(CH₂)_{N}-A-R, in which formula n is equal to at least 2, A an —OCO— or —COO— group and R a C₂₁-C₃₀ aliphatic group, more particularly those which have behenate functional groups —(CH₂)₃—O—CO—(CH₂)₂₀—CH₃ which are directly bonded to silicon atoms of the polyorganosiloxane chain, are very particularly advantageous for thickening some oily media.

A first subject of the invention consists of the use, for thickening oily media in which the main fatty phase is chosen from:

C₆-C₂₂, preferably C₁₄-C₂₀, saturated or unsaturated aliphatic fatty alcohols optionally containing 1 or more oxygen heteroatoms

ethers of polyethylene glycol or polypropylene glycol and of C₄-C₂₂, preferably C₁₀-C₂₀, fatty alcohols acetic esters of C₆-C₂₂, preferably C₁₄-C₂₀, saturated or unsaturated aliphatic fatty alcohols optionally containing 1 or more oxygen heteroatoms

aliphatic diesters of C₂-C₁₀, preferably C₂-C₈, dicarboxylic acids

natural or synthetic triglycerides

aliphatic isoparaffinic oils

paraffinic oils

optionally hydrogenated polyisobutene oils

volatile silicone oils

of silicone waxes containing aliphatic fatty alcohol or acid ester functional groups, of formula (I)

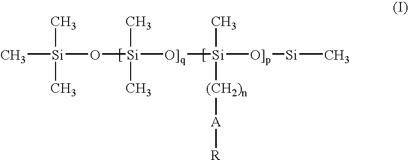

in which formula:

n denotes an integer equal to at least 2, preferably equal to 3

A denotes a

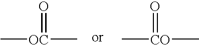

group

R denotes a C₂₁-C₃₀ aliphatic group

p denotes a whole or decimal number which can range from 1 to 100, preferably from 1 to 10,

q denotes a whole or decimal number which can range from 5 to 100, preferably from 10 to 100, the value of p/p+q being such that the number of

(CH₂)ₙ-A-R functional groups present in the wax of formula (I) is from approximately 50 to 250, preferably from approximately 100 to 250, milliequivalents per 100 g of the said wax,

the said waxes being chosen from those which have a melting point equal to at least 40° C.

The silicone waxes of formula (I) can be obtained by a hydrosilylation reaction of a polyhydroorganosiloxane of formula

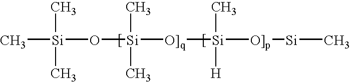

and of a compound of formula CH₂=CH₂—(CH₂)ₙ₋₂-A-R, n, p, q, A and R having the definition given above, in the presence of a known hydrosilylation catalyst like, for example, platinum complexes.

Among the preferred silicone waxes of formula (I) it is possible to mention very particularly silicone waxes containing behenate functional groups, which have the following formula (II):

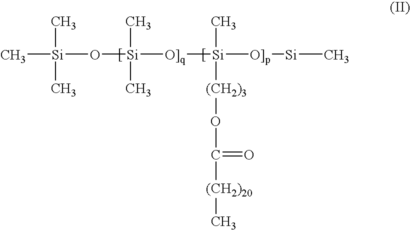

in which formula R denotes a whole or decimal number which can range from 1 to 100, preferably from 1 to 10, and q denotes a whole or decimal number which can range from 5 to 100, preferably from 10 to 100, the value of p/p+q being such that the number of behenate functional groups —(CH₂)₃—O—CO—(CH₂)₂₀—CH₃ present in the wax of formula (II) is from approximately 50 to 250, preferably from approximately 100 to 250, milliequivalents per 100 g of the said wax.

The waxes containing behenate functional groups, of formula (II), have a melting point higher than 40° C., generally of the order of 45 to 80° C.

The quantity of silicone wax of formula (I) or (II) which can be employed for thickening the said oily media may be of the order of 2 to 20, preferably of the order of 3 to 10, parts by weight per 100 parts by weight of the said oily media.

A fatty phase will be considered to be the main one in an oily medium when it represents of the order of at least 10%, preferably of at least 40% by weight of the said oily medium.

Volatile silicone oils are intended to mean cyclic or linear polydimethylsiloxanes containing from 3 to 9, preferably from 4 to 5 silicon atoms; they have a viscosity which is lower than approximately 20 mPa s at 25° C.

Among the fatty phases constituting the oily media that can be thickened thus, there may be mentioned especially

oleyl alcohol

isopropyl adipate

polypropylene glycol (PPG)-3 myristyl ether (Witconol APM® marketed by Witco), PPG-4 myristyl ether, PPG-4 lauryl ether or PPG-10 cetyl ether

mixtures of cetyl acetate and of acetylated lanolin alcohol (Crodalan LA® marketed by Croda)

caprylic acid triglyceride

corn oil

grapeseed oil

sweet almond oil

jojoba oil

squalane

optionally hydrogenated polyisobutenes

liquid paraffin

paraffinic oils such as liquid paraffin

isoparaffinic oils (Marchol 52® and Marchol 82® marketed by Esso)

volatile silicone oils such as hexamethylsiloxane, cyclomethicone D4 or cyclomethicone D5, by themselves or mixed with each other; up to 20% by weight of these volatile oils may be replaced by one or more silicone oils or silicone gums of high viscosity.

Oily media composed of cosmetic emollient mixtures like those which appear especially in “Cosmetics & Toiletries”, No. 93, page 107, July 1992, are particularly advantageous.

The said oily medium to be thickened may be present by itself or may constitute the oil phase of a “water-in-oil” or “oil-in-water” emulsion; it may also be present as such or in the form of a simple or multiple “water-in-oil” or “oil-in-water” emulsion within a cosmetic or pharmaceutical composition (cream, milk etc).

Another subject of the present invention is a process for thickening oily media in which the main fatty phase is chosen from

C₆-C₂₂, preferably C₁₄-C₂₀, saturated or unsaturated aliphatic fatty alcohols optionally containing 1 or more oxygen heteroatoms

ethers of polyethylene glycol or polypropylene glycol and of C₄-C₂₂, preferably C₁₀-C₂₀, fatty alcohols

acetic esters of C₆-C₂₂, preferably C₁₄-C₂₀, saturated or unsaturated aliphatic fatty alcohols optionally containing 1 or more oxygen heteroatoms

aliphatic diesters of C₂-C₁-C₁₀, preferably C₂-C₈, dicarboxylic acids

natural or synthetic triglycerides

aliphatic isoparaffinic oils

paraffinic oils

optionally hydrogenated polyisobutene oils

volatile silicone oils

by addition to the said media of at least one silicone wax containing aliphatic fatty alcohol or acid ester functional groups, of formula (I), which have a melting point of at least 40° C., preferably containing behenate functional groups, of formula (II) above, in a quantity of the order of 2 to 20, preferably of the order of 3 to 10, parts by weight per 100 parts by weight of oily medium to be thickened.

The preferred oily media which may be used are those already mentioned above.

The said process may be favourably carried out by mixing the oily medium to be thickened and wax containing aliphatic fatty alcohol or acid ester, preferably behenate, functional groups, at a temperature which is higher than that of the melting point of the wax; the said mixing may be carried out by any means that makes it possible to mix two fluids (gate paddle, tumbling, etc.). The mixture is allowed to cool without stirring; a gel or a thickened phase is thus obtained.

Another subject of the invention consists of a composition made up of an oily medium in which the main fatty phase is chosen from

C₆-C₂₂, preferably C₁₄-C₂₀, saturated or unsaturated aliphatic fatty alcohols optionally containing 1 or more oxygen heteroatoms

ethers of polyethylene glycol or polypropylene glycol and of C₄-C₂₂, preferably C₁₀-C₂₀, fatty alcohols

acetic esters of C₆-C₂₂, preferably C₁₄-C₂₀, saturated or unsaturated aliphatic fatty alcohols optionally containing 1 or more oxygen heteroatoms

aliphatic diesters of C₂-C₁₀, preferably C₂-C₈, dicarboxylic acids

natural or synthetic triglycerides

aliphatic isoparaffinic oils

paraffinic oils

optionally hydrogenated polyisobutene oils

volatile silicone oils

the said medium being thickened with the aid of the order of 2 to 20, preferably of the order of 3 to 10, % of its weight of at least one silicone wax of formula (I) which has a melting point of at least 40° C., or preferably of formula (II).

The preferred oily media which may be present are those already mentioned above.

The said composition may be made up of the said thickened oily medium by itself or of a simple or multiple “water-in-oil” or “oil-in-water” emulsion, in which emulsion the oil phase consists of the said thickened oily medium; the said composition may also be a cosmetic or pharmaceutical formulation containing the said thickened medium present as such or in the form of a simple or multiple “water-in-oil” or “oil-in-water” emulsion, beside other conventional additives for cosmetic or pharmaceutical formulations.

The silicone waxes containing ester or behenate functional groups, of formula (I) or (II), are very particularly advantageous for thickening the oily media part of the composition of sun gels or deodorants, especially for thickening the oily media containing more than 10%, preferably more than 50%, of their weight of volatile silicone oils in order to form translucent gels.

Deodorant compositions or those counteracting excessive perspiration (antiperspirants) are described in the literature. Such compositions generally contain silicones because of the sensory properties contributed by the latter. Among these properties there may be mentioned, for example, the volatile nature of cyclic silicones; the consequences of this volatility are the absence of a cold sensation after deposition on the skin, a nongreasy and nonsticky feel, and excellent lubricating behaviour. These properties, highly valuable for imparting a cosmetically acceptable character to the compositions, are transferred to the other constituents of these compositions, like the active constituents used in combination with these silicones. The silicones employed in these compositions are generally linear or cyclic polydimethylsiloxanes (described in the C.T.F.A.—Cosmetic, Toiletries, Fragrance Association—Dictionary, 5th Edition, 1993), like dimethicones, dimethiconols or cyclomethicones. Other types of silicones may also be present by themselves or in combination with those mentioned above, like those known under the names dimethicone copolyol, diphenyl dimethicone, phenyl dimethicone, amodimethicone etc. (CTFA dictionary).

In deodorant or antiperspirant compositions many other components are found, which are either active against perspiration or the formation of unpleasant odours, or modify the physical appearance of the compositions, depending on whether the final composition is in the form of a liquid, a gel or a solidified medium. Depending on their physical appearance and as a function of the degree of gelling that is chosen, these compositions may be in the form of sticks, extruded gels, roll-on applicators, creams, etc.

The application of the gel, of the thick or gelled cream or of the solidified medium to the skin is made pleasanter when the melting or softening temperature of the gel is close to skin temperature, which is reflected in a sliding effect during application.

Commonly used thickening, gelling or solidifying agents are lithium, sodium, potassium, aluminium, zirconium, cerium and similar salts, stearic, hydroxystearic, behenic and montanic acids, glycol, polyglycol, glycerol, polyglycerol and C₂-C₃₀ aliphatic alcohol esters of C₁₄-C₃₀ carboxylic or hydroxycarboxylic acids, polyethylene glycol or polypropylene glycol C₁₄-C₃₀ ethers, C₁₄-C₃₀ aliphatic alcohols etc.

Also to be found among the texture agents are conventional waxes which have melting points between 30° C. and 150° C., like beeswax, spermaceti, carnauba wax, paraffin wax, microcrystalline waxes, ceresin and ozokerite.

However, all these traditional solutions are generally not satisfactory for thickening the deodorant or antiperspirant compositions containing tetradimethylcyclosiloxane or pentadimethylcyclosiloxane as main volatile compound in the continuous phase, because of the generally experienced incompatibility of these compounds with silicones, whether volatile or otherwise. During the preparation of the composition with heating, at temperatures which are higher than the melting temperature of the waxes or of the thickening waxy media, this incompatibility is generally reflected in an impossibility of guaranteeing a homogeneous medium in the composition without maintaining energetic agitation. Sometimes this incompatibility may even result in a complete phase separation and demixing. After return to cold, this incompatibility when warm is reflected in the loss of the thickening properties or the formation of precipitates or agglomerates which are detrimental to the cosmetic appearance or feel of the composition (nonhomogeneity, hard bits, microcrystals etc.).

The silicone waxes of formulae (I) and (II) may be employed by themselves or in combination with waxy compounds or the waxes mentioned above as thickeners. In the case of the use in combination with other thickeners or gelling agents, the silicone waxes of formulae (I) and (II), besides their role as a thickener, also act as a compatibilizing agent between the conventional gelling agents and thickeners and the silicone-rich fatty phase. These silicone waxes also have the advantage of preventing the traditional waxes or waxy media from forming excessively coarse microcrystals which would give the composition an unpleasant feel. In this case the silicone waxes act as an inhibitor of the crystal growth by cocrystallizing with the other waxes and thus forming a network of microcrystallites which are finer than in the absence of any silicone wax. Furthermore, when employed alone or in combination with the other thickening waxes, these silicone waxes improve the cosmetic feel of the composition by decreasing the “greasy” feel and promoting spreading on the skin.

The active agents employed in the deodorant and/or antiperspirant compositions include astringent agents limiting perspiration, antibacterial agents, absorbent agents and additives controlling unpleasant odours, like 2,2′-bis(pyridine) disulphide described in EP-A-483428 or else the amino acids described in WO 9111998 or undecylenic acid.

Among the astringent compounds which restrict perspiration and which may be employed in the deodorant or antiperspirant compositions, there are to be found organic or inorganic aluminium, zirconium and zinc salts or their mixed salts or their mixtures. These compounds are described or mentioned in the literature, in particular in the journal Cosmetics & Toiletries, April 1990, pages 35 to 39. Examples of these antiperspirant compounds are aluminium chloride, aluminium and/or zirconium chlorohydrate, aluminium and/or zirconium dichlorohydrate, aluminium and/or zirconium trichlorohydrate, aluminium and/or zirconium tetratrichlorohydrate, aluminium and/or zirconium pentachlorohydrate, aluminium and/or zirconium sesquichlorohydrate, aluminium chlorohydrex, aluminium-zirconium chlorohydrex glycine, aluminium zirconium octachlorohydrate, aluminium sulphate, zinc sulphate, zirconium aluminium chlorohydroglycinate, zirconium hydroxychloride, zirconium aluminium lactate, aluminium potassium sulphate, aluminium sodium chlorohydroxylactate, aluminium bromohydrate, zinc sulphocarbonate, aluminium bromide, and zinc phenolsulphonate used in combination with aluminium sulphate.

In addition, all these astringent compounds may be encapsulated or protected by film-coating, adsorption, or complexing with polymers or by any other appropriate technique, like those described in U.S. Pat. No. 4,624,062.

Furthermore, bactericidal or bacteriostatic compounds may be added to these deodorant compositions to control the proliferation of the microbial flora which develops on the body. Among these compounds there may be mentioned chlorhexidine and its derivatives, nisine and trichlosan.

The persistence of these compounds on the skin may be improved, if desired, by addition of 0 to 10% by weight of polymers like polydimethylsiloxanes and polydimethyldiphenylsiloxanes of high viscosity, preferably higher than 100,000 mPa s.

The astringent compounds are generally present in concentrations of 1% to 70% by weight, preferably of 5 to 50% by weight.

The compounds controlling the proliferation of bacterial flora on the skin may be introduced in concentrations of 0.1 to 10%.

Besides all these compounds it is also possible to find in the deodorant compositions other volatile compounds like ethanol, isopropanol, emollients like those described in the journal Cosmetics & Toiletries, July 1992, No. 107, pages 93 et seq., or compounds like alkylmonoglycerides, alkyldiglycerides, diols like 1,2-propanediol, 1,3-butanediol, polyethylene glycols or polypropylene glycols and their C₂-C₁₂ esters, liquid fatty esters like isopropyl palmitate, 2-ethylhexyl cocoate and myristyl myristate, C₂-C₁₀ esters of isostearic acids or of C₂-C₈ hydroxycarboxylic acids, nonvolatile silicones like linear polyalkylsiloxanes, silicone copolyols or copolyethers etc, perfumes, moisturizing agents like glycerol, sorbitol, urea, collagen, aloe vera, hyaluronic acid, alcoxylated sugar derivatives or their esters like Glucam P200 or Glucam E20 which are marketed by Amerchol, and water. When the composition contains agents of marked hydrophilic nature, compatibilizing agents like anionic, nonionic, cationic or zwitterionic surfactants may be employed. Among these surfactants, those containing a polyorganosiloxane base structure are the preferred ones.

The use of silicones in the cosmetic compositions for sun protection has been known for a long time.

Cyclic volatile silicones like tetradimethylcyclosiloxane or pentadimethylcyclosiloxane are employed especially for their volatility, which allows fast drying of the composition when applied to the skin without being felt as a sensation of coldness. Furthermore, these volatile silicones impart the cosmetic properties sought after to the compositions by making the compositions easier to spread on the skin and by reducing the oily or greasy character of these compositions, particularly when they are formulated in the form of oil, of sun gel or of emulsion containing an oily continuous phase.

In general, silicones are also employed for their good emollient or skin-protective properties. These properties are described, for example, in a US Food and Drug Administration monograph (Department of Health and Human Services, Food and Drug Administration, Skin Protectant Drug Products for Over the Counter Human Use, 21 CFR Part 347).

The silicones best suited for their emollient properties are polydimethylsiloxanes, silicone copolyols, diphenyldimethicones, phenyltrimethicones, alkylsiloxanes and dimethiconols, of viscosities of between 20 and 10,000 mPa s.

Water resistance is essential for ensuring the persistence with time of the sun or anti-UV protection on the skin.

The use of polydimethylsiloxane of high molecular mass for ensuring better persistence of anti-UV agents on the skin is taught in EP-A-197485. In particular, polydimethylsilanes with a degree of polymerization higher than 5000 dimethylsiloxy units are appreciably resistant to washing.

Besides these additives, the cosmetic compositions which protect against the effects of UV radiation contain organic molecules acting as UV screens, or organic particles acting as a physical barrier to UV radiation. These UV screens or absorbents are well known in the literature; they are described, for example, in the paper in the journal Cosmetics & Toiletries, Vol. 102, March 1987, p.21 et seq. Examples which may be mentioned are UV screens like para-aminobenzoates and their derivatives, salicylates, cinnamates, benzophenones, benzylidene camphor, benzotriazoles and their derivatives and, in general, the screens mentioned in Appendix 7 of European Directive 76/768/EEC.

These anti-UV screens may additionally be grafted onto a polymer chain, in particular onto a polysiloxane chain.

Particles of titanium oxide, zinc oxide or cerium may be mentioned among the inorganic particles. These inorganic oxide particles or nanoparticles are optionally surface-coated with polymers, organic molecules or other inorganic compounds in order to improve their compatibility with the organic phases and to decrease their surface reactivity, such as photocatalysis.

The silicone waxes of formulae (I) and (II) may be introduced into sun protection compositions in concentrations of between 0.5% and 30% by weight of the composition, preferably from 1% to 20% by weight of this composition, in order to thicken or gel the hydrophobic organic phase of the composition, to improve the feel of these compositions by restricting the greasy feel and by making them easier to spread, to increase the persistence of the anti-UV active agents on the skin and in order to maintain, in time, the protective activity of the composition which is deposited on the skin.

The silicone waxes of formulae (I) and (II) may be combined with gelling or thickening compounds such as the abovementioned waxes or waxy media.

In addition to these compounds, the sun protection compositions may comprise emollients conventionally employed in cosmetics, perfumes, dyes, pigments or laquers, preserving agents, compounds which are active against oxidation or free radicals, skin hydrating agents and water.

The sun protection compositions may comprise the following compounds:

| | |
|---|---|
| Cyclomethicone | 0-80% |
| Other silicones | 0-10% |
| Volatile alcohols or ether-alcohols | 0-50% |
| (or other volatile compounds) | |
| Emollients, esters | 0-30% |
| Silicone wax of formula (I) | 0.5-15% |
| or (II) | |
| Other waxes or thickening systems | 0-10% |
| Anti-UV screen | 0-15% |
| Inorganic pigments | 0-10% |
| Water | 0-40% |
| Perfume, preserving agent | q.s. |

They may be prepared by mixing the constituents with agitation at a temperature which is higher than the melting points of the silicone wax and of the waxy media of the composition, and then pouring the composition into a suitable receptacle.

The following examples are given by way of illustration.

### EXAMPLE 1

10 g of the silicone wax containing behenate functional groups (B) defined below

wax of formula (II) in which p=2.5 and q=13, containing 80 meq. of behenic functional groups/100 g of polymer and exhibiting a melting point of 48.3° C.—

are incorporated into 90 g of an oily medium given in Table 1, by mixing with the aid of a gate paddle at a temperature of 60° C.

30 g of the mixture obtained are poured into a crystallizer 5 cm in diameter.

The mixture is allowed to cool to 20° C. for 24 hours.

It is found that, after cooling, the gel obtained does not flow when the crystallizer is turned over.

Comparative tests were carried out by replacing the wax (B) with the behenic silicone wax (B′) Abil Wax 2440 marketed by Goldschmitt, exhibiting a melting point of 42° C., and of formula

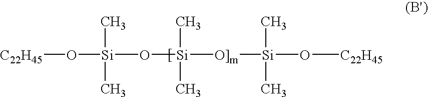

The results obtained are the following

| Oil | B | B′ |
|---|---|---|
| oleyl alcohol | gel | solution |
| isopropyl adipate | gel | incompatibility |
| Witconol APM ® | gel | solution |
| Crodalan LA ® | gel | solution |
| caprylic acid triglyceride | gel | solution |
| corn oil | gel | solution |
| grapeseed oil | gel | solution |
| sweet almond oil | gel | solution |
| jojoba oil | gel | solution |
| squalane | gel | solution |
| liquid paraffin | gel | solution |
| silicone oil D5 | gel | incompatibility |

### EXAMPLE 2

Deodorant formulation:

| | |
|---|---|
| Mirasil CM4 ® | 50% |
| (volatile cyclomethicone marketed by Rhone-Poulenc) | |
| Behenate silicone wax (B) | 18% |
| Hydrogenated castor oil | 5% |
| PEG-8 distearate | 2% |
| Aluminium zirconium tetrachlorohydrex-glyc. | 20% |
| (Reach AZP-908) | |
| Talc | 5% |

This formulation is prepared as follows.

The silicone wax is heated to 65° C. The volatile silicone is added with stirring and reflux and the other components are then introduced with stirring.

| | |
|---|---|
| Perfume | 1% |

The silicone wax is heated to 65° C. The other components are added under reflux and with stirring. After homogenizing, the mixture is cooled with stirring to 50° C. and poured into suitable moulds or receptacles.

### EXAMPLE 5

Nonaqueous sun gel formulation

| | |
|---|---|
| Mirasil CM5 ® | 39% |
| (cyclodimethicone marketed by Rhone-Poulenc) | |
| Benzophenone-3 | 3% |
| Isopropyl palmitate | 25% |
| Isononyl isononanoate | 25% |
| Behenate silicone wax (B) | 10% |

The various constituents are mixed at 65° C. under reflux with energetic stirring until the mixture is completely homogenized. After cooling with stirring to 50° C. the mixture is poured into a suitable receptacle.

The behenate silicone wax (B) thickens the formulation and contributes to its good spreading and to its nongreasy sensory character. It increases the persistence of the active constituents on the skin by reducing their water-sensitivity.

### EXAMPLE 6

Formulation of a sun gel

| | |
|---|---|
| Myritol 318 ® | 17.5% |
| (capryl caprylic triglyceride marketed by Henkel) | |
| Cetiol V ® | 7.8% |
| (decyl oleate marketed by Henkel) | |
| Mirasil CM4 ® | 9.8% |
| Diphenyl dimethicone | 10% |
| Liquid paraffin | 30.8% |
| Benzophenone-3 | 2% |
| Parsol MCX ® | 2% |
| (octyl methoxycinnamate marketed by Givaudan-Roure) | |
| Isostearic acid | 3.6% |
| Behenate silicone wax (B) | 10% |

The various constituents are mixed hot (65° C.) under reflux until completely homogenized. After cooling with stirring to 50° C., the mixture is poured into a suitable receptacle.

### EXAMPLE 7

Formulation of a gelled sun emulsion

| | |
|---|---|
| Phase A: | |
| Arlacel 1689 ® | 3.5% |
| (sorbitol and glycerol esters marketed by ICI) | |
| Liquid paraffin | 5.5% |
| Arlamol HD ® | 8.0% |
| (heptamethylnonane marketed by ICI) | |
| Tioveil TG ® | 8.0% |
| (dispersion of nanometre titanium oxide | |
| marketed by Tioxide) | |
| Parsol MCX ® | 2% |
| Behenate silicone wax (B) | 5.0% |
| Phase B: | |
| Glycerol | 4.0% |
| MgSO₄.7H₂O | 0.5% |
| Preserving agent | 0.5% |
| Water | q.s. 100 |

Phases A and B are prepared and mixed hot (75° C.) separately. Phase B is added slowly with stirring to phase A. After homogenization the composition is left to cool with stirring to 50° C. and is then transferred to a suitable receptacle.

The behenate silicone wax (B) contributes to thickening the composition and increases the persistence of the anti-UV compounds on the skin.

What is claimed is:

1. A process for thickening oily medium in which the main fatty phase is selected from the group consisting of:
C₆-C₂₂, saturated or unsaturated aliphatic fatty alcohols optionally containing 1 or more oxygen heteroatoms,
ethers of polyethylene glycol or polypropylene glycol and of C₄-C₂₂ fatty alcohols,
acetic esters of C₆-C₂₂ saturated or unsaturated aliphatic fatty alcohols optionally containing 1 or more oxygen heteroatoms,
aliphatic diesters of C₂-C₁₀ dicarboxylic acids,
natural triglycerides,
synthetic triglycerides,
aliphatic isoparaffinic oils,
paraffinic oils,
optionally hydrogenated polyisobutene oils, and
volatile silicone oils comprising the step of adding to said oily medium a thickening amount of at least one silicone wax containing aliphatic fatty alcohol or acid ester functional groups, of formula (I) 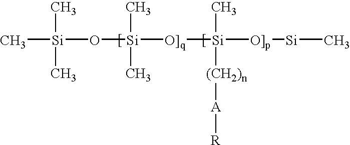
wherein:
n is an integer equal to at least about 2,
A is a 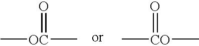
group
R is a C₂₁-C₃₀ aliphatic group
p is a whole or decimal number ranging from about 1 to about 100,
q is a whole or decimal number ranging from about 5 to about 100,
the value of p/p+q being such that the number of
—(CH₂)ₙ-A-R functional groups present in the wax of formula (I) is from about 50 to about 250 milliequivalents per 100 g of wax,
the said wax having a melting point equal to at least about 40° C.

2. A process according to claim 1 wherein the said silicone wax contains behenate functional groups and is represented by formula (II): 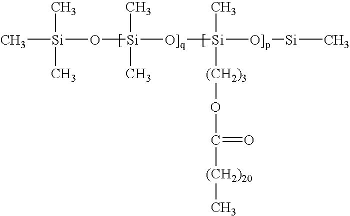
wherein p is a whole or decimal number ranging from about 1 to about 100, and q is a whole or decimal number ranging from about 5 to about 100, the value of p/p+q being such that the number of behenate functional groups —(CH₂)₃—O—CO—(CH₂)₂₀—CH₃ present in the wax of formula (II) being from about 50 to about 250 milliequivalents per 100 g of the said wax.

3. A process according to claim 2 wherein p is from about 1 to about 10 and q is about 10 to about 100.

4. A process according to claim 1 wherein the quantity of silicone wax of formula (I) or (II) added for thickening the said oily media is of about 2 to about 20 parts by weight per 100 parts by weight of the said oily medium.

5. A process according to claim 1 wherein the fatty phase constituting the oily medium to be thickened is selected from the group consisting of:
oleyl alcohol,
isopropyl adipate,
polypropylene glycol (PPG)-3 myristyl ether, PPG-4 myristyl ether, PPG-4 lauryl ether, PPG-10 cetyl ether,
mixtures of cetyl acetate and of acetylated lanolin alcohol,
caprylic acid triglyceride
corn oil,
grapeseed oil,
sweet almond oil,
jojoba oil,
squalane,
optionally hydrogenated polyisobutenes,
liquid paraffin,
paraffinic oils,
isoparaffinic oils,
volatile silicone oils,
hexamethyldisiloxane,
cyclomethicone D4, and
cyclomethicone D5.

6. A process according to claim 1 wherein the said oily medium to be thickened is present by itself.

7. A process according to claim 1 wherein the said oily medium constitutes the oil phase of a “water-in-oil” or “oil-in-water” emulsion.

8. A process according to claim 1 wherein the said oily medium to be thickened is in the form of a simple or multiple “water-in-oil” or “oil-in-water” emulsion within a cosmetic or pharmaceutical composition.

9. A process according to claim 8 wherein the said cosmetic composition is a sun gel or deodorant composition.

10. A process according to claim 9 wherein the said oily medium present within the sun gel or deodorant composition contains more than about 10%, of its weight of volatile silicone oils.

11. A process according to claim 10 wherein the said oily medium contains more than about 50% of its weight of volatile silicone oils.

12. A composition comprising an oily medium in which the main fatty phase is selected from the group consisting of:
—C₆-C₂₂ saturated or unsaturated aliphatic fatty alcohols optionally containing 1 or more oxygen heteroatoms,
ethers of polyethylene glycol or polypropylene glycol and of C₄-C₂₂ fatty alcohols,
acetic esters of C₆-C₂₂ saturated or unsaturated aliphatic fatty alcohols optionally containing 1 or more oxygen heteroatoms,
aliphatic diesters of C₂-C₁₀ dicarboxylic acids,
natural triglycerides
synthetic triglycerides,
aliphatic isoparaffinic oils,
paraffinic oils,
optionally hydrogenated polyisobutene oils,
volatile silicone oils,
said medium comprising from about 2 to about 20% of its weight of at least one thickening silicone wax containing aliphatic fatty alcohol or acid ester functional groups, of formula (I) 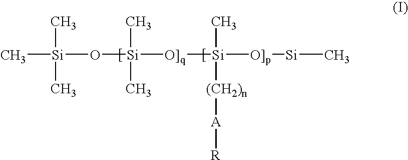
wherein:
n is an integer equal to at least about 2,
A is a 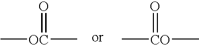
group
R is a C₂₁-C₃₀ aliphatic group
p is a whole or decimal number ranging from about 1 to about 100,
q is a whole or decimal number ranging from about 5 to about 100,
the value of p/p+q being such that the number of —(CH₂)ₙ-A-R functional groups present in the wax of formula (I) is from approximately about 50 to about 250, preferably from approximately about 100 to about 250, milliequivalents per 100 g of the said wax, the said wax being chosen from those which have a melting point equal to at least about 40° C.

13. A composition according to claim 12 wherein p is from about 1 to about 10 and q is from about 10 to about 100.

14. A composition according to claim 12 wherein said medium comprises about 3 to about 10% of its weight of at least one silicone wax of formula (I).

15. A composition according to claim 12 wherein the silicone waxe contains behenate functional groups and is represented by formula (II): 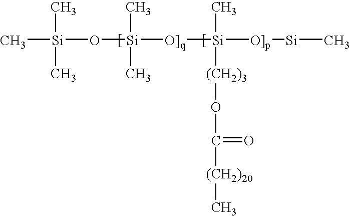
wherein p is a whole or decimal number ranging from about 1 to about 100, and is a whole or decimal number ranging from about 5 to about 100, the value of p/p+q being such that the number of behenate functional groups —(CH₂)₃—O—CO—(CH₂)₂₀—CH₃ present in the wax of formula (II) is from approximately about 50 to about 250 milliequivalents per 100 g of the said wax.

16. A composition according to claim 15 wherein p is from about 1 to about 10 and q is from about 10 to about 100.

17. A composition according to claim 15 wherein said medium comprises about 3 to about 10% of its weight of at least one silicone wax of formula (II).

18. A composition according to claim 12 wherein the fatty phase constituting the oily medium is selected from the group consisting of:
oleyl alcohol,
isopropyl adipate,
polypropylene glycol (PPG)-3 myristyl ether, PPG-4 myristyl ether, PPG-4 lauryl ether, PPG-10 cetyl ether
mixtures of cetyl acetate and of acetylated lanolin alcohol,
caprylic acid triglyceride,
corn oil,
grapeseed oil,
sweet almond oil,
jojoba oil,
squalane,
optionally hydrogenated polyisobutenes,
liquid paraffin,
paraffinic oils,
isoparaffinic oils,
volatile silicone oils,
hexamethylsiloxane,
cyclomethicone D4, and
cyclomethicone D5.

19. A composition according to claim 12 being in the form of a “water-in-oil” or “oil-in-water” emulsion, the oil phase of said emulsion consisting essentially of said oily medium thickened with said silicone wax.

20. A composition according to claim 12 being a cosmetic or pharmaceutical composition comprising the oily medium thickened with said silicone wax present as such.

21. A composition according to claim 12 being a cosmetic or pharmaceutical composition comprising the oily medium thickened with said silicone wax being in the form of a simple or multiple “water-in-oil” or “oil-in-water” emulsion.

22. A composition according to claim 20 wherein the said cosmetic composition is a sun gel or deodorant composition.

23. A composition according to claim 21 wherein the said cosmetic composition is a sun gel or deodorant composition.

24. A composition according to claim 22 wherein the said oily medium present within the sun gel or deodorant composition contains more than 10% of its weight of volatile silicone oils.

25. A composition according to claim 24 wherein the said oily medium present within the sun gel or deodorant composition contains more than 50% of its weight of volatile silicone oils.

26. A composition according to claim 23 wherein the said oily medium present within the sun gel or deodorant composition contains more than 10% of its weight of volatile silicone oils.

27. A composition according to claim 26 wherein the said oily medium present within the sun gel or deodorant composition contains more than 50% of its weight of volatile silicone oils.
